(19) United States
(12) Plant Patent Application Publication
(10) Pub. No.: US 2025/0386750 P1
(43) Pub. Date: Dec. 18, 2025

(54) Echinacea Plant Named 'BullEchipur 119'

(50) ;

(71) Applicant: Hartwig Bull, Gönnebek (DE)

(72) Inventor: Hartwig Bull, Gönnebek (DE)

(21) Appl. No.: 19/235,130

(22) Filed: Jun. 11, 2025

Related U.S. Application Data
(60) Provisional application No. 63/731,837, filed on Jun. 12, 2024.

(51) Int. Cl. A01H 6/14; A01H 5/02

(52) U.S. Cl. CPC A01H 6/1448; A01H 5/02
USPC PLT/428

(57) ABSTRACT

A new and distinct variety of Echinacea plant named ‘BullEchipur 119’ having large white flowers, good growth habit, uniform growth, good branching habit, and good propagation rates.

### CROSS REFERENCE TO RELATED APPLICATIONS

This application claims the benefit of priority from U.S. Provisional Patent Application No. 63/731,837, filed Jun. 12, 2024, which is incorporated herein by reference in its entirety.

Botanical classification: Echinacea purpurea.

Varietal denomination. ‘BULLECHIPUR 119’.

### BACKGROUND OF THE INVENTION

The present invention relates to a new and distinct cultivar of Echinacea, botanically known as Echinacea purpurea, and hereinafter referred to by the name ‘BullEchipur 119’.

The new Echinacea was discovered by the Inventor in August 2021, in a controlled environment in Gönnebek, Germany, as a part of a planned breeding program. The new Echinacea was selected on the basis of its large white flowers, good growth habit, uniform growth, good branching habit, and good propagation rates.

Asexual propagation of the new cultivar by in vitro culture since September 2021, in Gönnebek, Germany, has shown that the unique features of this new Echinacea are stable and reproduced true to type in successive generations.

### SUMMARY OF THE INVENTION

The following traits have been repeatedly observed and are determined to be unique characteristics of ‘BullEchipur 119’. These characteristics in combination distinguish ‘BullEchipur 119’ as a new and distinct cultivar:
1. Compact growth
2. White flowers
3. Lots of large flowers
4. More branching habit
5. Uniform growth
6. Nice green leaves
7. Fast crop time
8. Early flowering

In side-by-side comparisons conducted by the Inventor in Gönnebek, Germany, plants of the new Echinacea differed from plants of the female parent, ‘Bul-2020-05’ of Echinacea purpurea, in the following characteristics:
1. More compact
2. Larger white flowers
3. Good branching
4. Clean green leaves
5. Earlier flowering

In side-by-side comparisons conducted by the Inventor in Gönnebek, Germany, plants of the new Echinacea differed from plants of the male parent, ‘Bul-2020-09’ of Echinacea purpurea, in the following characteristics:
1. More compact
2. More vivid and larger flowers
3. More branches
4. Lots of flowers
5. Earlier flowering

Plants of the new Echinacea can be compared to the plants of the Echinacea cultivar ‘Pow Wow White’, believed to be unpatented. In side-by-side comparisons conducted by the Inventor in Gönnebek, Germany, plants of the new Echinacea differed from plants of the cultivar ‘Pow Wow White’ in the following characteristics:
1. More compact
2. More branching habit
3. Very even growth
4. More flowers
5. Nice cleaner leaves
6. Earlier flowering

### DESCRIPTION OF THE DRAWINGS

The accompanying colored photographs illustrate the overall appearance of the new Echinacea, showing the colors as true as it is reasonably possible to obtain in colored reproductions of this type. Colors in the photographs may differ slightly from the color values cited in the detailed botanical description, which accurately describes the colors of the new Echinacea.

FIG. 1 comprises a top perspective view of a typical plant of ‘BullEchipur 119’ under natural light.

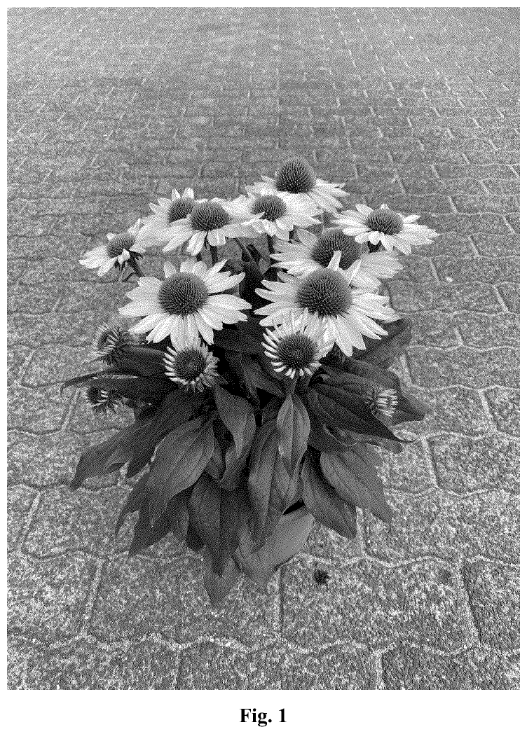

### DESCRIPTION OF THE PLANT

The cultivar Echinacea has not been observed under all possible environmental conditions. The phenotype may vary somewhat with variations in environment such as temperature and light intensity without, however, any variance in genotype.

The aforementioned photographs and following observations and measurements describe plants of the new Echinacea that were grown in an outdoor nursery in Gönnebek, Germany. During the production of the plants, temperatures averaged about 18-20° C. Plants used for the photographs and description were about 22 weeks from planting. Color references are made to The Royal Horticultural Society Colour Chart, 2015 version, except where general terms of ordinary dictionary significance are used.

### PLANT

Botanical classification: Echinacea purpurea.
Parentage: ‘Bul-2020-05’ and ‘Bul-2020-09’.
Propagation:
   Time to initiate roots.—About 7 days.
   Time to develop roots.—About 40-50 days.
   Time to produce a finished flowering plant.—About 12-14 weeks.
   Root description.—Lots of roots, free-branching, nice, healthy-looking root system.
Plant description:
   Growth rate/vigor.—Compact/upright.
   Plant height.—45-50 cm.
   Plant width.—35-40 cm.
   Branching habit.—Lots of branches, upright.
   Lateral branches.—Number per plant: 13-20. Length: 2-35 cm. Diameter: 3-8 mm. Angle: Upright, outward. Strength: Strong. Texture: Pubescent. Color: 146C. Internode length: 1-5 cm.
   Foliage.—Length: 3-20 cm. Width: 1-7 cm. Shape: Lanceolate. Shape of Apex: Acuminate. Shape of Base: Attenuate to Acute. Orientation and aspect: Straight to curved. Texture: Upper surface: Rough. Lower surface: Rough. Young leaves: Upper surface color: 137A. Lower surface color: 137C. Fully expanded leaves: Upper surface color: 137A. Lower surface color: 137C. Venation: Type: Cross venulate to longitudinal. Color: Upper surface: 145A. Lower surface: 145B. Margin: Entire.
Flowers:
   Bud.—Stage of opening: Single type blossom. Shape: Elliptic sharpened ray florets and tubular disk floret. Diameter: 1-3.5 cm. Length: 1.2-2.5 cm. Color: N137C.
   Natural Flowering Season.—10-12 weeks after planting.
   Inflorescence.—Form: Single flower. Disc and ray floret arrangement: Single composite flower. Average number per plant: 12-18. Average number per branch: 1-3. Fragrance: None Diameter: 4-10 cm. Height: 2-4 cm. Diameter of disc: 1.5-4 cm. Receptacle height: 1 cm. Receptacle diameter: 1.5-4 cm. Ray florets: Number per inflorescence: 15-22. Arrangement: In two to three whorls. Length: 1.5-4.4 cm. Width: 0.9-1.6 cm. Shape: Ellipse. Shape of Apex: Obtuse. Shape of Base: Acute. Margin: Entire. Texture: Upper surface: Smooth, glabrous. Lower surface: Rough. Color: When opening: Upper surface: 155C. Lower surface: 155C. At maturity: Upper surface: 155C. Lower surface: 155C. Ground color description: White.
   Disc florets.—Number per inflorescence: Numerous, too many to quantify. Arrangement: In center of receptacle. Length: 8-15 mm. Width: 1-2 mm. Shape: Tubular. Shape of Apex: Sharp. Color: Immature: 146C. At maturity: 153C. Phyllaries: Number per inflorescence: 35-50. Arrangement: In 3-4 whorls. Length: 0.5-1.5 cm. Width: 2-3 mm. Shape: Lanceolate. Shape of Apex: Elongate, oval. Shape of Base: Fused. Margin: Entire. Texture: Upper surface: Rough.

Lower surface: Rough. Color: Immature: Upper surface: NN137C. Lower surface: NN137C. At maturity: Upper surface: NN137C. Lower surface: NN137C.
Reproductive organs:
   Androecium.—Presence: On disk florets. Number: One. Filament: Length: 1-2 mm. Color: 153C. Anther: Shape: Oval. Length: 1 mm. Color: 153C. Pollen: Color: 15B. Amount: Too much to quantify.
   Gynoecium.—Presence: In disk and ray florets. Pistil length: 1-2 mm. Stigma: Shape: Two-parted. Color: 153C. Style: Length: 1-2 mm. Color: 153C.
   Seeds/fruit.—Length: 1-2 mm. Color:
Disease/pest resistance:
   Disease resistance.—No additional disease resistance has been observed beyond the typical resistance in Echinacea plants.
   Pest resistance.—No additional pest resistance has been observed beyond the typical resistance in Echinacea plants.
Weather tolerance:
   Temperature tolerance.—No additional temperature resistance has been observed beyond the typical resistance in Echinacea plants.
   Drought tolerance.—No additional drought resistance has been observed beyond the typical resistance in Echinacea plants.

I claim:

1. A new and distinct cultivar of Echinacea plant named ‘BullEchipur 119’, as illustrated and described.
